(19) United States
(12) Patent Application Publication
(10) Pub. No.: US 2002/0198518 A1
(43) Pub. Date: Dec. 26, 2002

(54) Entry position grid for computer guided cryosurgery

(76) Inventors: Paul W. Mikus, Irvine, CA (US); Jay J. Eum, Irvine, CA (US); Wilson S. Wong, Alhambra, CA (US)

Correspondence Address: Lawrence N. Ginsberg, Endocare, Inc., 201 Technology Drive, Irvine, CA, 92618, US

(21) Appl. No.: 10/120,722

(22) Filed: Apr. 11, 2002

Related U.S. Application Data
(63) Continuation-in-part of application No. 09/699,938, filed on Oct. 30, 2000.
Application No. 09/699,938 is a continuation of application No. 09/318,710, filed on May 26, 1999, now Pat. No. 6,139,544.
(60) Provisional application No. 60/283,576, filed on Apr. 13, 2001.

(51) Int. Cl. A61B 18/18

(52) U.S. Cl. 606/21; 600/439

(57) ABSTRACT

An entry position grid for aiding a user in locating the approximate entry points of a plurality of cryoprobes into a patient, wherein the cryoprobes are inserted into the perineal area of the patient and then into the prostate, the entry position grid including a frame portion having a center longitudinal axis, a grid portion extending across the frame portion, the grid portion defining a plurality of evenly spaced discrete openings, identifying indicia located on the frame that identifies each discrete opening, each discrete opening being adapted to allow the user to position the cryoprobes through the entry position grid and into the patient at an angle that is not parallel to the center axis, the grid portion comprising a continuous strand of flexible material that is attached to the frame portion.

This application is a continuation-in-part patent application of U.S. application Ser. No. 09/699,938 filed on Oct. 30, 2000.

### BACKGROUND OF THE INVENTION

Cryosurgical probes are used to destroy living tissue by thermally manipulating a cryoprobe to extreme temperatures by using freezing and heating cycles. Current cryosurgical probes are incorporated into computer guided cryosurgery systems wherein the system calculates the optimal placement for the cryoprobes into diseased tissue. However, current computer guided cryosurgical systems restrict the user to a single insertion angle. As a result, collateral and healthy tissue may be frozen and damaged as a result of the current insertion angle limitations of the cryoprobes.

Therefore, what is needed is a computer guided cryosurgical apparatus, system, and method that allow the user to introduce cryosurgical probes into the patient with a wide variety of insertion angles.

### FIELD OF THE INVENTION

The invention is directed to cryosurgery and, more particularly, to a device used with computer guided cryosurgery.

### SUMMARY OF THE INVENTION

The present invention relates to an apparatus, system, and method for aiding a user in locating the approximate entry points of a plurality of cryoprobes into a patient, wherein the cryoprobes are inserted into the perineal area of the patient and then into the prostate.

In accordance with one aspect of the present invention, an apparatus for aiding a user in locating the approximate entry point of at least one cryoprobe into the patient includes a frame portion having a center axis, a grid portion extending across the frame portion, with the grid portion defining a plurality of evenly spaced discrete openings, and identifying indicia corresponding to the plurality of spaced discrete openings, with each discrete opening being adapted to allow the user to position the cryoprobe at an angle that is not parallel, or non-parallel, to the center axis.

In accordance with an additional aspect of the present invention, a system for aiding the user and locating the approximate entry point of at least one cryoprobe into the patient during a cryosurgical procedure, includes at least one cryoprobe for insertion into the patient, and an entry position grid, which includes a frame portion having a center axis, a grid portion disposed internally of the frame portion, a grid portion defining a plurality of evenly spaced discrete openings, and identifying indicia corresponding to the discrete openings, and each discrete opening being adapted to allow the user to position the cryoprobe at an angle that is not parallel to the center axis of the frame portion.

Further, in accordance with an additional aspect of the present invention, a method for aiding a user in locating the approximate entry point of a cryoprobe into the patient during a cryosurgical procedure, includes providing at least one cryoprobe, and providing an entry position grid, which includes a frame portion having a center axis, a grid portion disposed internally of the frame portion, a grid portion defining a plurality of evenly spaced discrete openings, and identifying indicia corresponding to the discrete openings, each discrete opening being adapted to allow the user to position the cryoprobe at an angle that is not paralleled to the center axis, the method further including the steps of displaying on a computer screen an image of the entry position grid and an image of the desired entry points of the cryoprobe into the patent in relation to the entry position grid.

A further aspect of the present invention includes the method of performing a cryosurgical procedure on a patient, which includes inserting an imaging probe into the patient's rectum to gather data on the patient's prostate, providing the data to a computer system for processing, the computer system calculating the desired placement of at least one cryoprobe into the patient's prostate based on the data gathered from the imaging probe, the computer system displaying on a computer screen a desired entry point of the cryoprobe into the patient image in relation to an entry position grid image, aligning the cryoprobe in relation to an entry position grid, and inserting at least one cryoprobe into the patient through the entry position grid, the entry position grid including a frame portion having a center axis, a grid portion disposed internally of the frame portion, the grid portion defining a plurality of evenly spaced discrete openings, and identifying indicia corresponding to the discrete openings, wherein each discrete opening is adapted to allow the user to position the cryoprobe at an angle that is not parallel to the center axis of the frame portion.

In accordance with an additional aspect of the present invention, an apparatus for aiding a user in locating the approximate entry point of at least one cryoprobe into the patient includes a frame portion having a center axis, a grid portion extending across the frame portion, a grid portion defining a plurality of evenly spaced discrete openings, and identifying indicia corresponding to the plurality of spaced discrete openings, each discrete opening being adapted to allow the user to position the cryoprobe at an angle that is not parallel to the center axis, the grid portion being comprised of a flexible material so that the grid portion is flexible in relation to the frame portion.

Further, in accordance with an additional aspect of the present invention, an entry position grid for aiding a user in locating the approximate entry points of a plurality of cryoprobes into a patient, wherein the cryoprobes are inserted into the perineal area of the patient and then into the prostate, includes a frame portion having a center longitudinal axis, a grid portion extending across the frame portion, the grid portion defining a plurality of the evenly spaced discrete openings, identifying indicia located on the frame that identifies each discrete opening, each discrete opening being adapted to allow the user to position the cryoprobes through the entry position grid into the patient at an angle that is not parallel to the center axis, the grid portion being flexible in relation to the frame portion.

In accordance with an additional aspect of the present invention, an entry position grid for aiding a user in locating the approximate entry point of a plurality of cryoprobes into a patient, wherein the cryoprobes are inserted into the perineal area of the patient and then into the prostate, the entry position grid includes a frame portion having a center longitudinal axis, a grid portion extending across the frame portion, the grid portion defining a plurality of evenly spaced discrete openings, identifying indicia located on the frame that identifies each discrete opening, each discrete opening being adapted to allow the user to position the cryoprobes through the entry position grid and into the patient at an angle that is not parallel to the center longitudinal axis of the frame portion, the grid portion comprising a flexible material that is attached to the frame.

Further, in accordance with an additional aspect of the present invention, an entry position grid for aiding a user in locating the approximate entry points of a plurality of cryoprobes into a patient, wherein the cryoprobes are inserted into the perineal area of the patient and then into the prostate, the entry position grid includes a frame portion having a center longitudinal axis, a grid portion extending across the frame portion, the grid portion defining a plurality of evenly spaced discrete openings, identifying indicia located on the frame that identifies each discrete opening, each discrete opening being adapted to allow the user to position the cryoprobes through the entry position grid and into the patient at an angle that is not parallel to the center longitudinal axis of the frame portion, the grid portion including a continuous strand of flexible material that is attached to the frame.

A further aspect of the present invention includes an entry position grid for aiding a user and locating the approximate entry point of a plurality of cryoprobes into a patient, wherein the cryoprobes are inserted into the perineal area of the patient and then into the prostate, the entry position grid includes a frame portion having a center longitudinal axis, a grid portion extending across the frame portion, the grid portion defining a plurality of evenly spaced discrete openings, identifying indicia located on the frame that identifies each discrete opening, each discrete opening being adapted to allow the user to position the cryoprobes through the entry position grid and into the patient at an angle that is not parallel to the center longitudinal axis of the frame portion, the grid portion comprising a continuous strand of suture material that is attached to the frame portion.

Further, in accordance with an additional aspect of the present invention, an entry position grid for aiding a user in locating the approximate entry point of a plurality of cryoprobes into a patient, wherein the cryoprobes are inserted into the perineal area of the patient, and then into the prostate, the entry position grid includes a frame portion having a center longitudinal axis, a grid portion extending across the frame portion, the grid portion defining a plurality of evenly spaced discrete openings, identifying indicia located on the frame portion that identifies each discrete opening, each discrete opening being adapted to allow the user to position the cryoprobes through the entry position grid and into the patient at an angle that is not parallel to the center axis, a grid portion being comprised of a flexible material, so that the grid portion is flexible in relation the frame portion.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a cross-sectional view of a cryosurgical procedure according to the present invention;

FIG. 2 is a perspective view of an incremental stepper, ultrasound probe, and entry position grid according to the present invention;

FIG. 3 is an illustration of a longitudinal cross-sectional view of the prostate as displayed on a monitor by the system according to the present invention;

FIG. 4 is an illustration of a horizontal cross-sectional view of the prostate, an image of the entry position grid, and an image of an entry point as displayed by the system according to the present invention;

FIG. 5 is an illustration of the longitudinal cross-sectional view of the prostate, the image of the entry position grid, and an image of a cryosurgical probe as displayed by the system according to the present invention;

FIG. 6 is an exploded perspective view of a first embodiment of the entry position grid according to the present invention;

FIG. 7 is a perspective view of an entry position grid according to the present invention;

FIG. 8 is a front elevational view of the entry position grid according to the present invention;

FIG. 9 is a front elevational view of an entry position grid according to a second embodiment of the present invention;

FIG. 10 is an elevational side view of the entry position grid according to the second embodiment; and

FIG. 11 is a rear elevational view of the entry position grid according to the second embodiment.

### DETAILED DESCRIPTION OF THE PREFERRED EMBODIMENTS

The present invention relates to an apparatus, system, and method for aiding a user and locating the approximate entry points of a plurality of cryoprobes into a patient, wherein the cryoprobes inserted into the perineal area of the patient and then into the prostate. The apparatus according to the present invention is an entry position grid that allows the user a full range of insertion angles of the cryoprobes so that the cryoprobes may be inserted into the patient at a wide variety of angles in relation to the entry position grid.

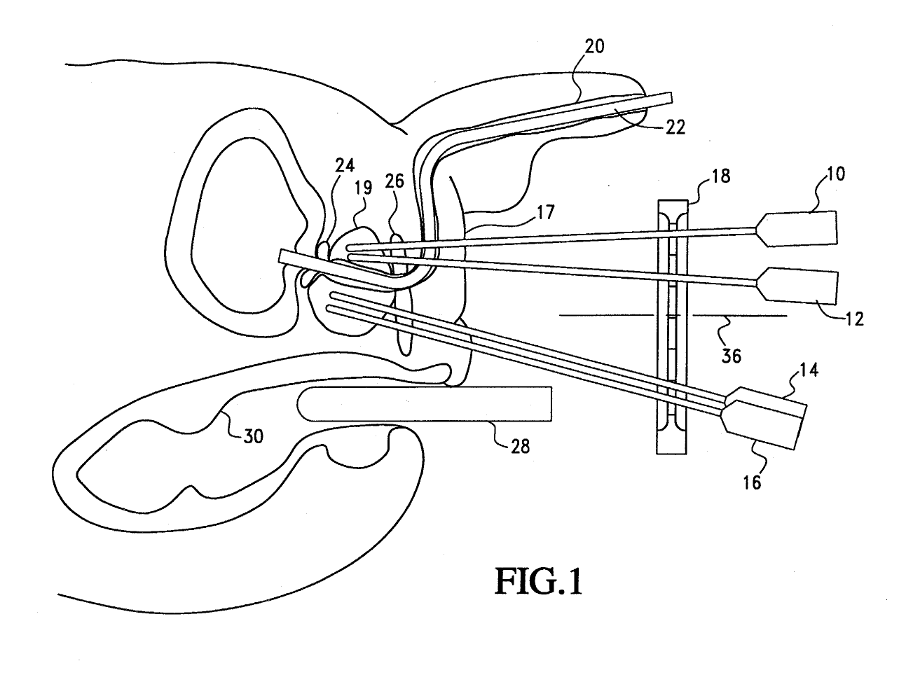

Referring to FIG. 1, a cryosurgical procedure according to the present invention is shown. A plurality of cryoprobes 10, 12, 14, and 16 are shown inserted into the patient's perineal region 17 and then into the patient's prostate 19. The urethra 20, which passes through the prostate, is one of the anatomic structures that usually should not be frozen during the cryosurgical procedure. Accordingly, the urethra 20 is protected and kept warm with a urethra warming catheter 22. The bladder neck sphincter 24 and the external sphincter 26 are also structures that should be protected from freezing by the warming catheter 22. Neurovascular bundles on the right and the left sides of the prostate are also protected from freezing by the angle of insertion of the cryoprobes which is provided for by the entry position grid according to the present invention. Transrectal probe 28 is inserted into the rectum 30 in order to visualize the placement of the cryoprobes and the growth of the ice balls formed by the cryoprobes. To assist in placement of the cryoprobes, an entry position grid 18 is used as described in more detail below to aid the user in locating the approximate entry points of the cryoprobes into the patient at the perineal area. Further, an image is produced and displayed as a 2-dimensional representation and/or a 3-dimensional representation illustrating the boundaries of the prostate. Images are also provided by the system to provide real time feedback of the placement of the cryoprobes.

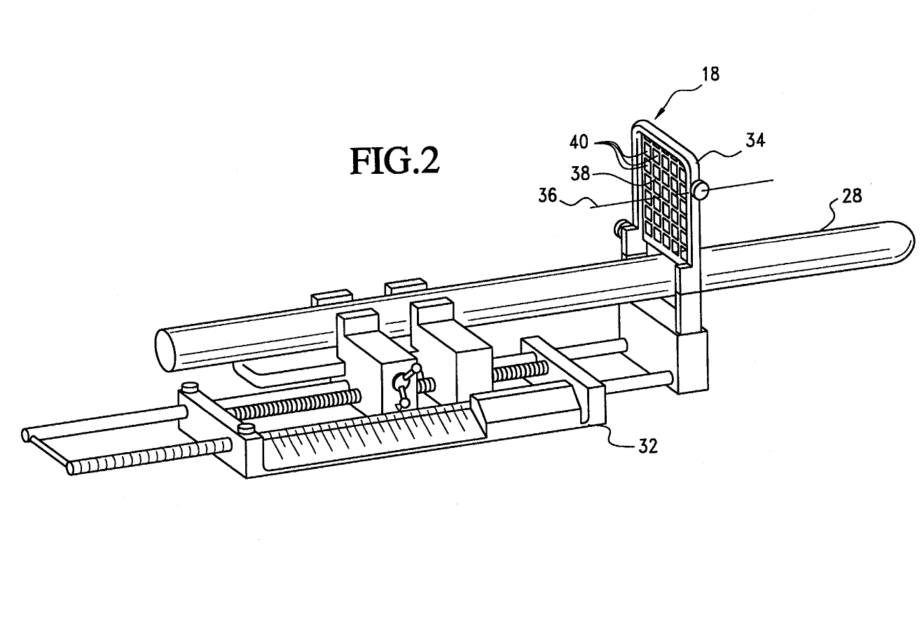

FIG. 2 illustrates an incremental stepper 32 with the ultrasound probe 28 detachably fixed thereto. The incremental stepper 32 includes the entry position grid 18 which includes a frame portion 34 having a center longitudinal axis 36 which is parallel with the ultrasound probe 28. Further, the entry position grid 18 includes a grid portion 38 extending across the frame portion 34 and defining a plurality of evenly spaced discrete openings 40. The incremental stepper 32 is secured to the table that the patient is lying on. Then, the entry position grid 18 is backed away from the perineal area of the patient. The next step is to advance the ultrasound probe 28 into the patient's rectum so as to capture a full image of the patient's prostate. The ultrasound probe 28 is incrementally backed out of the patient's rectum by retracting the ultrasound probe 28 in 5 mm increments as provided for by the incremental stepper 32. Images of the prostate are created at each incremental step and processed through a computer system so that images may be created of the prostate at each incremental image taken. This imaging process and corresponding system is disclosed in co-pending U.S. application Ser. No. 09/699,938 filed on Oct. 30, 2000, which is incorporated herein in its entirety as if set out in whole and is included herein as Attachment 1. Next, after the images are taken of the prostate, the entry position grid 18 is advanced to be positioned against or adjacent to the patient's perineal area to aid the user in locating the approximate entry points of the cryoprobes into the patient.

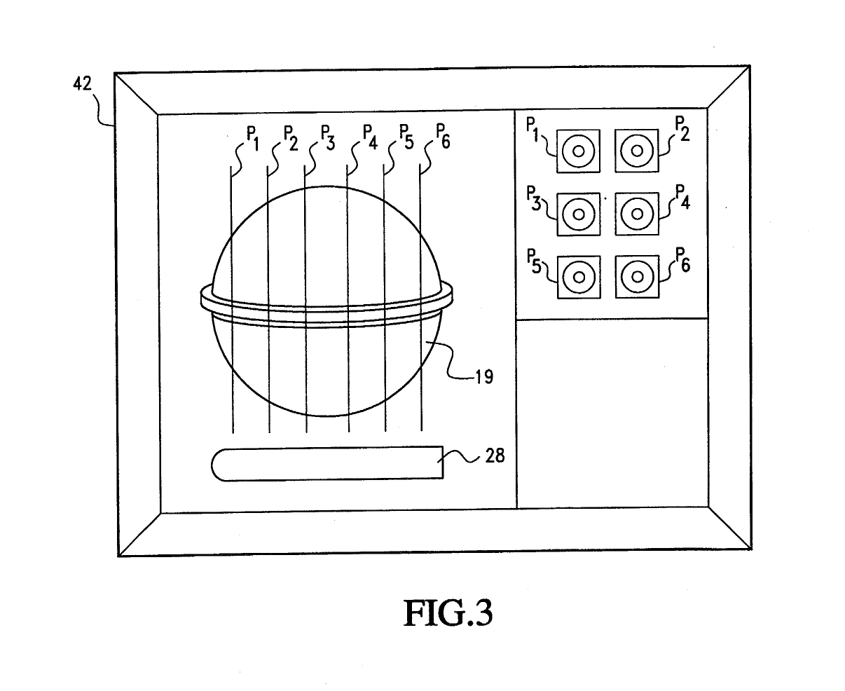

Referring to FIG. 3, images are shown on a display 42, wherein the ultrasound probe 28 has taken 6 images of the prostate at positions P1, P2, P3, P4, P5, and P6. In a separate area of the display 42, the longitudinal cross-sectional images are displayed individually as images of the prostate at corresponding positions P1, P2, P3, P4, P5, and P6.

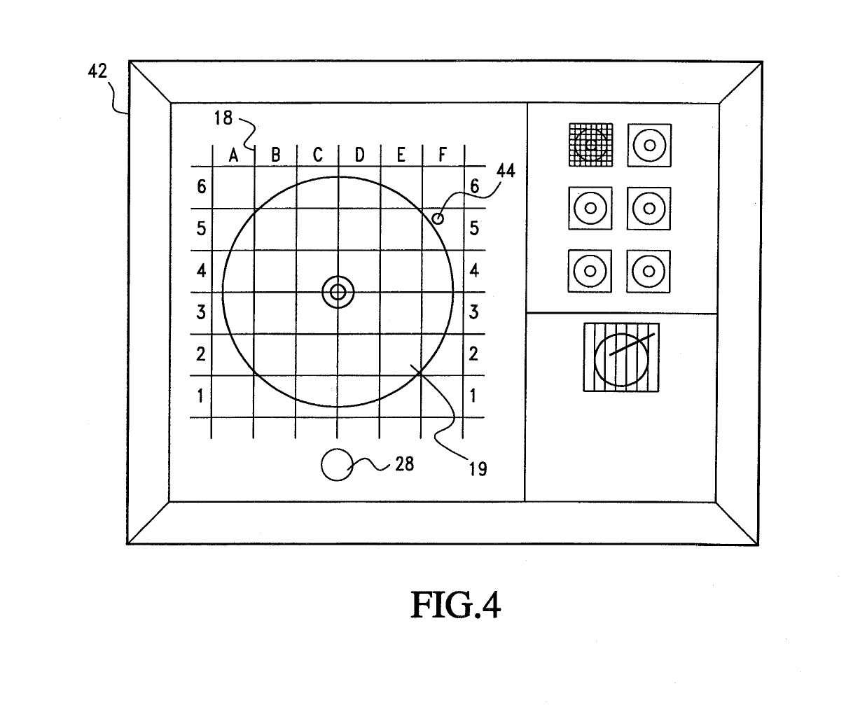

As shown in FIG. 4, a horizontal view of the prostate 19 is shown with an overlay of the entry position grid 18. Also shown in the display 42 is a calculated entry position point 44 which is overlaid with the image of the entry position grid 18. The entry position point 44, as shown in this example, is calculated to be in the upper left portion of discrete opening 40 in the entry position grid at location F5.

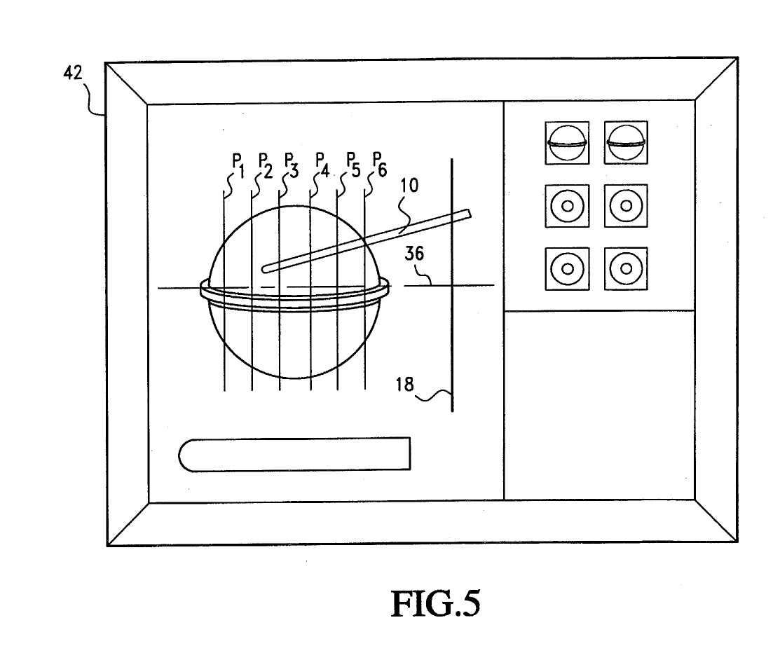

FIG. 5 illustrates placement of cryoprobe 10 into the prostate through the entry position grid 18 and through planes P3, P4, P5, and P6 at an angle that is not parallel with the entry grid position center longitudinal axis 36. The user may freely switch between the images on the display 42 from the longitudinal cross-section view of the prostate at any one of the positions P1 through P6 to the horizontal cross-sectional view of the prostate at any of the portions P1 through P6. In addition, since real time images are provided of the prostate and the placement of the cryoprobes into the prostate, the user is provided real time feedback regarding the actual placement of the cryoprobe into the prostate versus the calculated and intended placement of each cryoprobe into the prostate. Therefore, the user aligns the cryoprobe in relation to the entry position grid, then inserts the cryoprobe into the perineal region of the patient, and then into the prostate of the patient by viewing a series of overlaid images of a placement grid created for each position P1 through P6, depending upon the depth of the intended cryoprobe placement into the patient. For example, as the user inserts the cryoprobe into the entry position grid 18 and advances it toward the prostate, the user would then select the image at position P6 which would include an overlaid image of a position grid at P6 and a target point for the cryoprobe. For example, the target point of the cryoprobe tip at position P6 may be, for example, at discrete opening E4 in relation to entry point 44 at discrete opening F5. The entry position grid 18 of the present invention allows for placement of cryoprobes into the patient at a variety of angles in relation to the center longitudinal axis 36. Therefore, certain anatomical structures, such as the urethra or the rectum, may be avoided and not frozen during the cryosurgical procedure based on the wide range of angles provided for by the entry position grid 18. Further, placement of the cryoprobes into the prostate and the resulting ice balls from the cryosurgical procedure may be maximized since the cryoprobes according to the present invention may be placed into the prostate at certain angles that would maximize the anatomical structure or shape of a particular patient's prostate.

It will be appreciated by those skilled in the art that the entry position grid, the system, and the methods disclosed herein may be utilized with any cryosurgical procedure performed on a patient and the utility of such should not be limited to the cryosurgical procedure upon a patient's prostate.

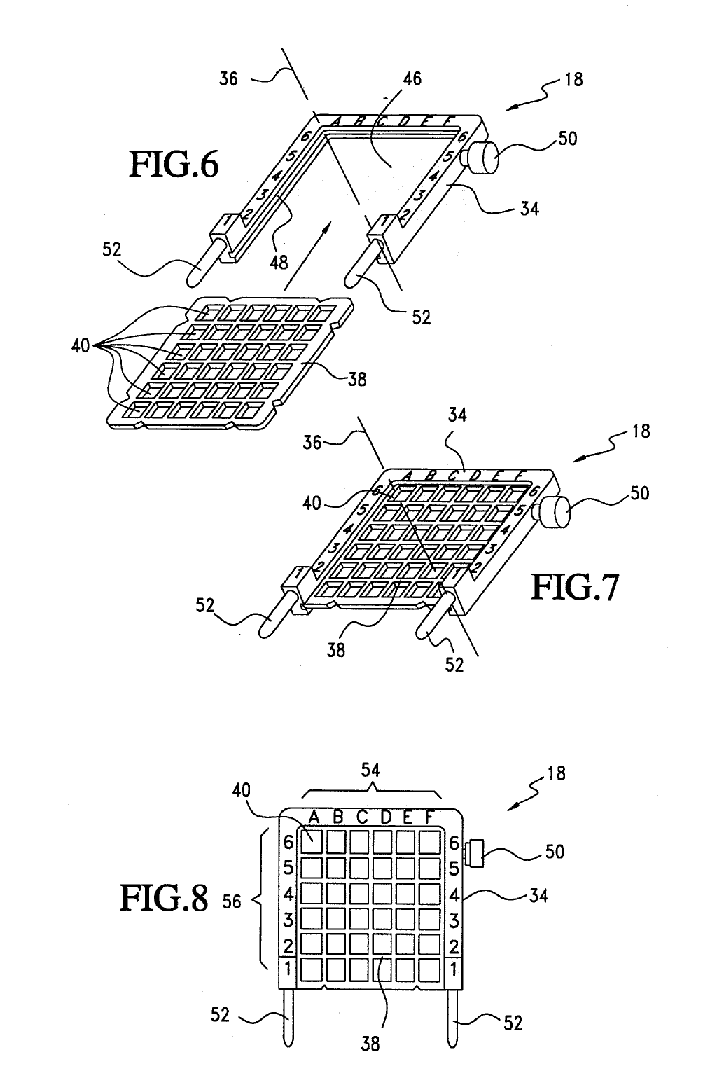

A first embodiment of the entry position grid according to the present invention is disclosed in FIGS. 6, 7, and 8. FIG. 6 illustrates an entry position grid 18, which includes a frame portion 34 having a center longitudinal axis 36 and a frame opening 46. The frame portion also includes a track 48 to allow for a grid portion 38 to be slidably inserted into the frame portion 34. The grid portion 38 is securedly attached to the frame portion 34 by a set screw 50. The frame portion further includes two locating pins 52, which are used to locate the entry position grid on the incremental stepper 32. FIG. 8 further identifies identifying indicia, including horizontal indicia 54 and vertical indicia 56, wherein the identifying indicia corresponds to the plurality of spaced discrete openings 40. In the preferred embodiment, each discrete opening is approximately 5 mm wide×5 mm in height, which allows the user to position the cryoprobes at any angle of entry into the patient, whether the angle is parallel to or nonparallel the center longitudinal axis 36 of the entry position grid 18. This freedom of movement by the user of the cryoprobe is further illustrated in the fact that typically cryoprobe diameters are approximately 2 mm in diameter. Therefore, for example, the placement of a 2 mm diameter cryoprobe into a 5 mm×5 mm opening would allow the user to manipulate the cryoprobe to a wide variety of angles in relation to the center longitudinal axis. In addition, the entry position grid 18 is only 1.5 mm thick so as to allow for a wide range of positioning angles of the cryoprobes.

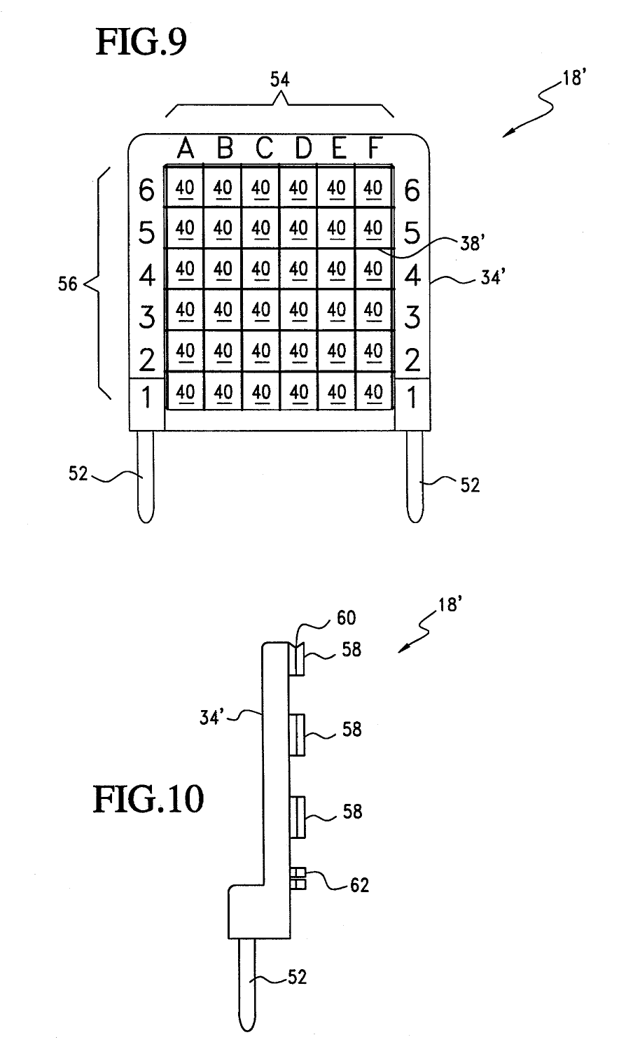

FIG. 9 illustrates another exemplary embodiment according to the present invention wherein like parts will bear similar reference numerals as in the first disclosed embodiment. The second embodiment is similar to the first disclosed embodiment except that the grid portion is comprised of a continuous strand of flexible material, such as No. 2 suture material, as commonly used in medical procedures. The suture material is threaded onto the frame portion so that the grid portion is flexible in relation to the frame portion 34′. Further, each strand of the grid portion 38′ is moveable in relation to the other strand portions of the grid portion 38′ so that if the user is required to place a cryoprobe at an entry point 44 which lies on a strand portion, the user may move the strand portion so that the cryoprobe may be accurately placed at the desired and calculated entry point 44. Since the strand portions are pulled at their ends by the frame portion, the moved strand portion returns to its original position after the strand portion is adjusted by the user.

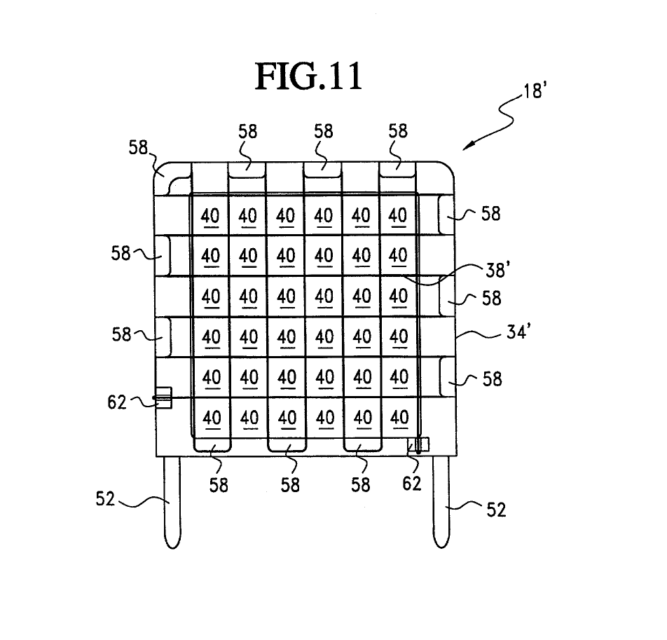

FIG. 9 shows an entry position grid 18′ with a frame portion 34′ and a grid portion 38′. FIG. 10 and FIG. 11 illustrate elevated side and rear views, respectively, of the entry position grid 18′ with a plurality of protrusions 58 extending from the rear side of the frame portion 34′. In FIG. 10, the grid portion is removed from the frame portion 34′ to illustrate the configuration of the protrusions 58. The protrusions 58 include a centered indentation 60 so that when the continuous strand is wound and secured onto the frame portion 34′, the strand is centered and seated properly onto the protrusions 58. As shown in FIG. 11, the protrusions 58 are offset from each other on the frame portion 34′ to allow a continuous strand to be wound across the frame portion 34′ to create a grid portion 38′. The continuous strand is tied off at two locations at the beginning of the strand and the end of the strand at tie off protrusions 62.

Further embodiments of the entry position grid according to the present invention may include a grid portion that is made of any material that would allow one strand of the grid portion to be moved by the user in relation to an adjacent discrete opening 40. Examples of the material that may be used to comprise the grid portion include an elastomeric material, such as rubber, a polypropylene material, such as fishing line, a cotton material, such as thread, or any other material that would be suitable for such purposes. Another advantage of utilizing a continuous strand to comprise the grid portion is that after the cryosurgical procedure is accomplished, the continuous strand is severed and removed from the frame portion and disposed, while the frame portion is sterilized and prepared for the next cryosurgical procedure.

It will further be appreciated by those skilled in the art that a variety of embodiments may be utilized to comprise a grid portion that is attached to the frame portion. For example, a grid portion may be comprised of a clear plastic material, such as cellophane, which is printed with a grid that corresponds to the identifying indicia on the frame portion with the cellophane grid portion being attached to the frame portion and then disposed of after each use. Another example of a grid portion includes a frame portion that has a plurality of light emitting diodes (LED) that emit light across the frame portion opening to comprise the grid portion and to define the discrete openings.

Although this invention has been shown and described with respect to detailed embodiments, those skilled in the art will understand that various changes in form and detail may be made without departing from the scope of the claimed invention.

1. An apparatus for aiding a user in locating the approximate entry point of at least one cryoprobe into the patient, comprising:
a frame portion having a center axis;
a grid portion extending across the frame portion, the grid portion defining a plurality of evenly spaced discrete openings; and
identifying indicia corresponding to the plurality of spaced discrete openings;
each discrete opening being adapted to allow the user to position the cryoprobe at an angle that is not parallel to the center axis.

2. The apparatus of claim 1, wherein the identifying indicia is located on the frame.

3. The apparatus of claim 2, wherein the identifying indicia includes a combination of letters and numbers.

4. The apparatus of claim 1, wherein the identifying indicia identifies each spaced discrete opening.

5. The apparatus of claim 1, wherein the plurality of spaced discrete openings are evenly spaced.

6. The apparatus of claim 1, wherein the apparatus is used for a cryosurgical procedure on the patient's prostate and the apparatus is adapted to be placed adjacent to the patient's perineal area and at least one cryoprobe is inserted through the apparatus and through the patient's perineal area.

7. The apparatus of claim 1, wherein said discrete openings have widths in a range of from about 5 mm to about 20 mm.

8. The apparatus of claim 1, wherein said discrete openings have heights in a range of from about 5 mm to about 20 mm.

9. The apparatus of claim 1, wherein said discrete openings have widths in a range of from about 5 mm to about 20 mm and have heights in a range of from about 5 mm to about 20 mm.

10. The apparatus of claim 1, said evenly space discrete openings are approximately 10 mm wide and approximately 10 mm in height.

11. The apparatus of claim 1, wherein said entry position grid is about 1.5 mm thick.

12. The apparatus of claim 1, wherein said evenly spaced discrete openings are approximately 5 mm in width by approximately 5 mm in height.

13. A system for aiding a user in locating the approximate entry point of at least one cryoprobe into the patient during a cryosurgical procedure, comprising:
at least one cryoprobe for insertion into the patient; and
an entry position grid, said entry position grid, comprising:
a frame portion having a center axis;
a grid portion disposed internally of the frame portion, said grid portion defining a plurality of evenly spaced discrete openings; and
identifying indicia corresponding to the discrete openings,
each discrete opening being adapted to allow the user to position the cryoprobe at an angle that is not parallel to the center axis.

14. A method for aiding a user in locating the approximate entry point of a cryoprobe into the patient during a cryosurgical procedure, comprising:
providing at least one cryoprobe;
providing an entry position grid, comprising:
a frame portion having a center axis;
a grid portion disposed internally of the frame portion, the grid portion defining a plurality of evenly spaced discrete openings; and,
identifying indicia corresponding to the discrete openings,
each discrete opening being adapted to allow the user to position the cryoprobe at an angle that is not parallel to the center axis; and,
displaying on a computer screen an image of the entry position grid and an image of the desired entry point of the cryoprobe into the patient in relation to the entry position grid.

15. A method of performing a cryosurgical procedure on a patient, comprising:
inserting an imaging probe into the patient's rectum to gather data on the patient's prostate;
providing the data to a computer system for processing;
the computer system calculating the desired placement of at least one cryoprobe into the patient's prostate based on the data from the imaging probe;
the computer system displaying on a computer screen a desired entry point of the cryoprobe into the patient image in relation to an entry position grid image;
aligning the cryoprobe in relation to an entry position grid; and
inserting at least one cryoprobe into the patient through the entry position grid;
the entry position grid comprising:
a frame portion having a center axis;
a grid portion disposed internally of the frame portion, the grid portion defining a plurality of evenly spaced discrete openings; and
identifying indicia corresponding to the discrete openings,
each discrete opening being adapted to allow the user to position the cryoprobe at an angle that is not parallel to the center axis.

16. An entry position grid for aiding a user in locating the approximate entry points of a plurality of cryoprobes into a patient, wherein the cryoprobes are inserted into the perineal area of the patient and then into the prostate, the entry position grid comprising:
means for defining a plurality of evenly spaced discrete openings that span across a frame having a center longitudinal axis;
means for identifying each discrete opening on the frame;
each discrete opening being adapted to allow the user to position the cryoprobes through the entry position grid and into the patient at an angle that is not parallel to the center longitudinal axis.
